(19) United States
(12) Patent Application Publication
(10) Pub. No.: US 2020/0132976 A1
(43) Pub. Date: Apr. 30, 2020

(54) LUMINESCENCE IMAGING APPARATUS AND METHODS

(71) Applicant: KING'S COLLEGE LONDON, Strand, London (GB)

(72) Inventors: Simon Morris AMEER-BEG, London (GB); Simon POLAND, London (GB); James LEVITT, London (GB); Jakub NEDBAL, London (GB)

(21) Appl. No.: 16/627,956

(22) PCT Filed: Jul. 3, 2018
(86) PCT No.: PCT/GB2018/051865

§ 371 Date: Dec. 31, 2019

(30) Foreign Application Priority Data
Jul. 4, 2017 (GB) 1710743.4

(51) Int. Cl. G02B 21/00; G01N 21/64; A61B 5/00

(52) U.S. Cl. CPC G02B 21/0076; G02B 21/0032; A61B 5/0071; G01N 21/6458; G02B 21/0036

(57) ABSTRACT

Luminescence imaging apparatus, methods and computer program products are disclosed. A time-resolved luminescence imaging apparatus (100A) comprises: an optical assembly (2) operable to generate an array of beams; a scanner (4A) operable to scan the array of beams with respect to a sample (8), along a single scanning axis; and a detector assembly (10) having an array of detector elements, adjacent detector elements being spaced apart by an inter-element gap, each detector element being operable to detect emissions generated by the sample (8) in response to the array of beams. In this way, different locations on the sample (8) may be simultaneously scanned and imaged by the detector assembly (10) in order to image multiple parts of the sample (8) simultaneously. Also, by scanning along a single scanning axis, the complexity of the scanner (4A) is significantly reduced and the speed of scanning is increased compared to scanners which have to scan in two dimensions, such as a traditional raster scan mechanism.

### FIELD OF THE INVENTION

The present invention relates to luminescence imaging apparatus, methods and computer program products.

### BACKGROUND

Imaging apparatus are known. In the field of microscopy, microscopes are used to image objects in areas of objects that cannot normally be seen with the naked eye. Different types of microscopes exist which provide beams which interact with a specimen, together with the collection of scattered beams from the specimen in order to create an image. Some specimens can have compounds attached to certain parts of the specimen which undergo fluorescence or luminescence under different circumstances, such as due to an interaction between parts of the specimen, which can be detected. Such imaging will typically be carried out by wide field irradiation of the sample or by scanning a beam over the sample. Such techniques have particular applicability in imaging of cells in order to understand the composition and interaction of components of those cells. Although these techniques are useful, many have their own shortcomings. Accordingly, it is desired to provide improved techniques for imaging.

### SUMMARY

According to a first aspect, there is provided a time-resolved luminescence imaging apparatus, comprising: an optical assembly operable to generate an array of beams; a scanner operable to scan the array of beams with respect to a sample, along a single scanning axis; and a detector assembly having an array of detector elements, adjacent detector elements being spaced apart by an inter-element gap, each detector element being operable to detect emissions generated by the sample in response to the array of beams.

The first aspect recognises that a problem with existing imaging apparatus is that they are complex and the image acquisition time can be orders of magnitude slower than interactions which are desired to be investigated. For example, the first aspect recognises that existing approaches may take many minutes to image a cell (or a portion thereof) whereas many dynamic biological events occur on significantly faster timescales. Hence, this time limitation can make the detection or observation of such biological events difficult or even impossible.

Accordingly, a luminescence imaging apparatus may be provided. The imaging apparatus may be time-resolved. The imaging apparatus may comprise an optical assembly. The optical assembly may generate, emit or provide an array or group of beams. The beams may be photon beams. The apparatus may comprise a scanner. The scanner may scan, move or translate the beams with respect to a sample. The scanner may scan the beams along a single, elongate or linear scanning axis. The imaging apparatus may comprise a detector assembly. The detector assembly may have an array of detector elements. Adjacent or neighbouring detector elements may be spaced apart or separated by an inter-element gap. Each detector element may detect emissions generated or produced by the sample in response to the array of beams. Such emissions may be photon emissions. In this way, different locations on the sample may be simultaneously scanned and imaged by the detector assembly in order to image multiple parts of the sample simultaneously. Also, by scanning along a single scanning axis, the complexity of the scanner is significantly reduced and the speed of scanning is increased compared to scanners which have to scan in two dimensions, such as a traditional raster scan mechanism.

In one embodiment, a diameter of each detector element is less than the inter-element gap. Accordingly, the diameter or field of view of each detector element may be less than the gap between detector elements.

In one embodiment, each detector element has a fill factor of less than 50%. Hence, the imaging area of each detector element may be less than half of the total area occupied by the detector element. That is to say that the detector elements are spaced apart.

In one embodiment, each detector element is operable to perform time-correlated single photon counting.

In one embodiment, each detector element comprises a single-photon avalanche diode.

In one embodiment the scanner is operable to scan the array of beams over the sample only along the single scanning axis. Accordingly, the scanner may scan the array of beams with respect to the sample only, just or solely along the unitary, elongate, one-dimensional scanning axis.

In one embodiment, the scanner is operable to scan the array of beams, each beam providing a scan line over the sample along the scanning axis. Accordingly, each beam may follow or define only a single scan line with respect to the sample, along the scanning axis.

In one embodiment the array of excitation beam elements comprise beam elements arranged in rows, extending along a beam row axis and in columns, extending along a beam column axis and the scanning axis is orientated between the row axis and the column axis. Accordingly, the array of excitation beam elements may be arranged in rows and columns. The scanning axis may be orientated or aligned to extend between the row axis and the column axis. That is to say that the scanning axis fails to align with either the row axis or the column axis in order that every excitation beam element in a row or every excitation beam element in a column does not follow the same scan line of the sample. This approach enables excitation beam elements to scan different scan lines with respect to the sample in order to cover spatially more of the sample than would be possible should the scan lines follow the row axis or the column axis.

In one embodiment, the scanning axis is orientated to scan the array of beams to provide non-overlapping scan lines on the sample. Providing non-overlapping scan lines maximises the spatial amount of sample imaged by the combined detector elements.

In one embodiment, the scanning axis is orientated to scan the array of beams to provide uniformly separated scan lines. Providing uniformly separated scan lines helps to uniformly distribute the scanning across the sample.

In one embodiment, the scanning axis is orientated to scan the array of beams to provide variably separated scan lines. Hence, the distribution of the separate scan lines may be varied.

In one embodiment, the scanning axis is orientated to scan the array of beams to provide scan lines separated by a distance of no less than half of a selected spatial resolution. It will be appreciated that for Nyquist sampling, 2×highest resolvable frequency is required. It is perfectly acceptable to over-sample, but it is not strictly required to achieve the best resolution.

In one embodiment, the scanning axis is orientated to scan the array of beams to provide overlapping scan lines on the sample. Allowing some scan lines to overlap enables the data generated by sub-optimal detector elements to be compensated for or disregarded since emissions from the same parts of the sample are collected at different times by different detector elements.

In one embodiment, the scanner is operable to set the scan axis to a selected orientation with respect to the sample. Accordingly, the orientation of the scanner axis may be selectable.

In one embodiment, the apparatus comprises logic operable to provide an indication of an orientation of the scanning axis. Accordingly, an indication of the orientation of the scanning axis may be provided in order to facilitate the spatial reconstruction of the data from the detector elements.

In one embodiment, the scanner comprises an optical scanner operable to direct the array of beams over the sample, along the scanning axis. Accordingly, the scanner itself may move the beams over the sample along the scanning axis.

In one embodiment, the scanner comprises a sample positioner operable to move the sample to direct the array of beams over the sample, along the scanning axis. Accordingly, the scanner may move the sample in order to move the beams over the sample along the scanning axis.

In one embodiment, the sample positioner is operable to orientate a conduit, through which the sample is conveyed, along the scanning axis.

In one embodiment, each detector element detects photons generated by the sample in response an associated beam. Accordingly, each detector may detect the photons or other emissions generated by the sample in response to a beam.

In one embodiment, the apparatus comprises processing logic operable to generate a sample image from detection data provided by each detection element in response to detected emissions.

In one embodiment, the processing logic is operable to generate the sample image using the indication of the orientation of the scanning axis.

In one embodiment, the processing logic is operable to generate the sample image by interpolating the scan lines to generate unscanned portions of the sample image. Using interpolation helps to reconstruct the missing spatial regions of the image.

In one embodiment, the processing logic is operable to compensate for detector element variation using overlapping scan lines when generating the sample image. Typically, the data from malfunctioning detector elements may be disregarded and data from a correctly functioning detector element which follows the same scan line may be used instead.

In one embodiment, the processing logic is operable to disregard data generated by detector elements exhibiting greater than a selected variation, such as greater than an selected signal to noise ratio.

In one embodiment, the processing logic is operable to generate temporally-separated sample images using overlapping scan lines. Accordingly, a sequence of images may be generated from the overlapping scan lines. This is because the detector elements that follow the same scan line detect emissions from the same locations at different times.

In one embodiment, the processing logic is operable to determine a speed at which the array of beams scan over the sample. By determining the speed at which the beams scan with respect to the sample, the collected data can be collated in order to prevent blurring.

In one embodiment, the processing logic is operable to determine the speed in response to at least one of an indication of a movement speed of the optical scanner and an indication of a sample speed determined from successive sample images. Accordingly, the speed may be determined from the scanner or from the movement of elements of the sample in successive sample images.

In one embodiment, the processing logic is operable to vary a number of detector emissions used to generate each pixel of the sample image in response to the sample speed.

In one embodiment, the array of detector elements comprises an ‘n’×‘m’ array of detector elements.

In one embodiment, the array of detector elements comprises an ‘n’×‘n’ array of detector elements.

In one embodiment, the array of beams comprises beams arranged in rows and in columns.

In one embodiment, each beam has a diffraction-limited beam width.

In one embodiment, a spacing between beams is proportional to the inter-element gap.

In one embodiment, the array of beams is arranged to illuminate the sample and the detector assembly is arranged orthogonally with respect to the array of beams to detect emissions from said sample.

According to a second aspect, there is provided a time-resolved luminescence imaging method, comprising: generating an array of beams; scanning the array of beams with respect to a sample, along a single scanning axis; and detecting emissions generated by the sample in response to the array of beams with an array of detector elements of a detector assembly, adjacent detector elements being spaced apart by an inter-element gap.

In one embodiment, a diameter of each detector element is less than the inter-element gap.

In one embodiment, each detector element has a fill factor of less than 50%.

In one embodiment, the method comprises performing time-correlated single photon counting with each detector element.

In one embodiment, each detector element comprises a single-photon avalanche diode.

In one embodiment, the array of beams comprise beams arranged in rows, extending along a beam row axis and in columns, extending along a beam column axis and the method comprises orientating the scanning axis between the beam row axis and the beam column axis.

In one embodiment, the method comprises scanning the array of beams over the sample only along the single scanning axis.

In one embodiment, the method comprises scanning the array of beams, each beam providing a scan line over the sample along the scanning axis.

In one embodiment, the method comprises orientating the scanning axis to scan the array of beams to provide non-overlapping scan lines on the sample.

In one embodiment, the method comprises orientating the scanning axis to scan the array of beams to provide uniformly separated scan lines.

In one embodiment, the method comprises orientating the scanning axis to scan the array of beams to provide variably separated scan lines.

In one embodiment, the method comprises orientating the scanning axis to scan the array of beams to provide scan lines separated by a distance of no less than half of a selected spatial resolution.

In one embodiment, the method comprises orientating the scanning axis to scan the array of beams to provide overlapping scan lines on the sample.

In one embodiment, the method comprises setting the scan axis to a selected orientation with respect to the sample.

In one embodiment, the method comprises providing an indication of an orientation of the scanning axis.

In one embodiment, the method comprises directing the array of beams over the sample, along the scanning axis with an optical scanner.

In one embodiment, the method comprises moving the sample with a sample positioner to direct the array of beams over the sample, along the scanning axis.

In one embodiment, the method comprises orientating a conduit, through which the sample is conveyed, along the scanning axis.

In one embodiment, the method comprises detecting photons generated by the sample in response an associated beam with each detector element.

In one embodiment, the method comprises generating a sample image from detection data provided by each detection element in response to detected emissions.

In one embodiment, the method comprises generating the sample image using the indication of the orientation of the scanning axis.

In one embodiment, the method comprises generating the sample image by interpolating the scan lines to generate unscanned portions of the sample image.

In one embodiment, the method comprises compensating for detector element variation using overlapping scan lines when generating the sample image.

In one embodiment, the method comprises disregarding data generated by detector elements exhibiting greater than a selected variation.

In one embodiment, the method comprises generating temporally-separated sample images using overlapping scan lines.

In one embodiment, the method comprises determining a speed at which the array of beams scan over the sample.

In one embodiment, the method comprises determining the speed in response to at least one of an indication of a movement speed of the optical scanner and an indication of a sample speed determined from successive sample images.

In one embodiment, the method comprises varying a number of detector emissions used to generate each pixel of the sample image in response to the sample speed.

In one embodiment, the array of detector elements comprises an ‘n’×‘m’ array of detector elements.

In one embodiment, the array of detector elements comprises an ‘n’×‘n’ array of detector elements.

In one embodiment, the array of beams comprises beams arranged in rows and in columns.

In one embodiment, each beam has a diffraction-limited beam width.

In one embodiment, a spacing between beams is proportional to the inter-element gap.

In one embodiment, the method comprises arranging the array of beams to illuminate the sample and arranging the detector assembly orthogonally with respect to the array of beams to detect emissions from said sample.

According to a third aspect, there is provided a computer program product operable, when executed on a computer to control an imaging apparatus to perform the method of the second aspect.

According to a fourth aspect, there is provided an imaging apparatus, comprising: a detector assembly having at least one detector element operable to detect photon emissions generated in response to optical stimulation by a sample to be imaged; and processing logic operable to identify fluorescing molecules within the sample by identifying differences in detected emission decay rates occurring within different regions of the sample detected by the detector assembly.

The fourth aspect recognises that super-resolution and localization fluorescence microscopy techniques have attracted considerable attention in the past decade in particular as they allow for localization of fluorophores on length scales below the optical diffraction limit, and elucidation of nanometre scale structural features in biological samples. The techniques can be broadly separated into three categories—(1) Photo-activation and photo-switching of molecules to image small subsets of individual emitters sequentially, frame-by frame, followed by reconstruction of the entire image from individual frames, (2) spatio-temporal manipulation of interacting laser beams to modify the excited state emission of fluorophores whilst either the laser beams or the sample are scanned and (3) structured illumination using patterned excitation light.

Whilst these methods allow for visualisation of biological structures with very high spatial resolution, close to or at the level of single molecules, uncovering the underlying biological function and dynamics of the system under study still represents a major challenge. Artefacts and uncertainties in localization microscopy also exist due to the stochastic nature of fluorophore blinking and switching. In addition, multi-colour experiments which would be advantageous for monitoring colocalization of proteins labelled with different fluorophores, for example, suffer from complications due to chromatic aberrations in imaging systems, leading to compromised localization precision. A problem in super-resolution microscopy is the persistence of fluorescence from molecules in successive frames during the acquisition which leads to an uncertainty in the number and position of molecules. Furthermore, measuring the number of molecules in a cluster of emitters mutually spaced at a distance much shorter than the optical resolution is also problematic. There are methods available, including single-molecule high-resolution imaging with photobleaching and processing algorithms. However, the available algorithms are limited and generally require sparse distributions of emitters within an image to achieve high accuracy. Generally, if a number of identical emitters are present within an area defined by a point spread function (PSF), and are emitting simultaneously then asserting the number and position of those emitters is non-trivial.

The vast majority of super-resolution microscopy techniques rely only on spatio-temporal variations in fluorescence intensity as the contrast mechanism by which a complete image can be reconstructed. Individual molecules are activated or switched such that only a sparse subset of all the molecules in the sample emit in any one frame of acquisition. The fluctuations in intensity can be routinely measured using sensitive cameras. However, fluorescence can be described by many parameters including the fluorescence lifetime, the average time spent in the excited state before emission. Indeed, of the fluorescence microscopy techniques capable of probing dynamic interactions, e.g. protein-protein interactions, fluorescence lifetime imaging (FLIM) is both well-established and very powerful. In the event that two neighbouring emitters are interacting via Förster resonance energy transfer (FRET), then the measured decrease in the fluorescence lifetime of the “donor” molecule can provide a measure of proximity to and degree of interaction with the “acceptor” molecule.

In one known arrangement, fluorescence is measured from populations of two dyes with very similar emission spectra deposited on a microscope coverslip, and localised single molecules by using prior knowledge of the individual fluorescence lifetimes of the two dyes to first generate intensity images for populations of each type of dye. A major benefit of using the fluorescence lifetime as the main contrast parameter is that affords the possibility to distinguish between molecules with similar or identical emission spectra, allowing more than one fluorescent label to be used without 10l introducing the localization problems associated with chromatic aberrations. Furthermore, the fluorescence lifetime is sensitive to changes in the local surroundings of a fluorophore with a read-out that is largely independent of concentration. In another known arrangement, the time-variant emission PSF was measured using a continuous wave STED beam.

The fourth aspect also recognises that a problem with existing imaging techniques is that while they can provide an image of a sample, those images provide little information about any functional interactions occurring within the sample. Accordingly, an imaging apparatus is provided. The imaging apparatus may comprise a detector assembly. The detector assembly may have one or more detector elements. Each detector element may detector photon emissions which are generated by a sample to be imaged. Such emissions may be generated in response to or following optical (typically photon) stimulation of that sample. The imaging apparatus may comprise processing logic. The processing logic may identify fluorescing molecules within the sample. The processing logic may identify those fluorescing molecules by identifying or analysing differences in the decay rates detected by the detector assembly which occur for fluorescing molecules in different regions of the sample. That is to say that the photon emissions detected by the detector assembly have an associated decay rate. Fluorescing molecules in different regions of the sample may exhibit different decay rates. The differences in those decay rates may identify the presence of different fluorescing molecules within the sample. In this way, not only can the sample be imaged but functional information identifying fluorescing molecules within the sample can also be identified.

In one embodiment, each detector element is operable to detect photon emissions generated over a detection time period in response to the optical (photon) stimulation. Accordingly, the detector element may detect photon emissions occurring over a selected period of time in response to the stimulation. It will be appreciated that by collecting data on the photon emissions detected over that period of time, a decay rate of those emissions can also be determined over that period of time.

In one embodiment, the fluorescing molecule comprises molecules undergoing FRET interactions. Accordingly, the processing logic may be operable to identify those fluorescing molecules which are undergoing FRET interaction.

In one embodiment, the processing logic is operable to provide an indication of FRET efficiency based on a magnitude of the differences in the detected photon emission decay rates within the different regions of the sample. Accordingly, depending on the magnitude or size of the differences in the decay rates between different regions of the sample, an indication of the efficiency or degree to which FRET interactions occur can be provided.

In one embodiment, the processing logic is operable to identify differences in the detected photon emission decay rates occurring over each detection time period within the different regions of the sample.

In one embodiment, the processing logic is operable to identify differences in the detected photon emission decay rates by comparing integrated emissions detected over each detection time period in the different regions.

In one embodiment, the processing logic is operable to identify differences in the detected photon emission decay rates by determining a change in a centre of mass of integrated emissions detected within each detection time period across the different regions. Accordingly, the integrated emissions within each detection time period may be analysed to determine a centre of mass and examine how the location of that centre of mass changes within that detection time period in order to identify the differences in the photon emission decay rates.

In one embodiment, the processing logic is operable to provide an indication of FRET efficiency based on a rate of change of the centre of mass. Accordingly, the FRET efficiency may be proportional to the rate of change of the location of centre of mass.

In one embodiment, the different regions are neighbouring regions. Hence, the regions may neighbour or be adjacent or near each other.

In one embodiment, the neighbouring regions are at least partially overlapping regions. Accordingly, part of one region may be included in another region.

In one embodiment, the processing logic is operable to identify differences in the detected photon emission decay rates by utilising integrated emissions detected over each detection time period in each at least partially overlapping region to provide a spatially-correlated intensity point spread function. Accordingly, the detected photon emissions for each sampling location may be combined for neighbouring or overlapping regions to give a spatially correlated intensity point spread function which can be used to identify differences in the decay rates to provide an indication of the activity of fluorescing molecules.

In one embodiment, the processing logic is operable to identify FRET interactions from an asymmetry in the spatially-correlated intensity point spread function. A lack of symmetry in the point spread function may provide an indication of FRET interactions.

In one embodiment, the processing logic is operable to identify differences in the detected photon emission decay rates by utilising integrated emissions detected over subsets of each detection time period in each at least partially overlapping region to provide at least one spatially-correlated intensity point spread function. Accordingly, the different sub-periods within the detection period at each sampling location may be used to provide different intensity point spread functions for those sub-periods in order to help identify interactions.

In one embodiment, the processing logic is operable to spatially locate a source of the emissions by fitting one or more emission curves to each spatially-correlated intensity point spread function, a centre of mass of each fitted emission curve spatially locating a source of the emissions. Accordingly, curves may be fitted to the point spread function and the location of each of those fitted curves may provide an indication of the location of fluorescing molecules being stimulated.

In one embodiment, the processing logic is operable to identify FRET interactions from a change in a centre of mass of each spatially-correlated intensity point spread function over each subset of the detection time period.

In one embodiment, the processing logic is operable to spatially locate a source of FRET interactions from the change in the centre of mass. The change in the location of the centre of mass may provide an indication of the source of the FRET interactions.

In one embodiment, the processing logic is operable to spatially locate the source of FRET interactions by extrapolating the change in the centre of mass. Accordingly, if the decay has not fully completed within the detection period, a trajectory of the movement of the centre of mass can still be determined and extrapolated in order to identify the location of the FRET interactions.

In one embodiment, each subset of the detection time period comprises a different subset of the detection time period.

In one embodiment, each subset of the detection time period comprises overlapping subsets of the detection time period.

In one embodiment, each subset of the time period iteratively excludes one of later and earlier detected photon emission within the detection time period.

In one embodiment, the processing logic is operable to identify differences in the detected photon emission decay rates by identifying deviations from a predefined decay rate over each detection time period in each region.

In one embodiment, the processing logic is operable to provide an indication of FRET efficiency based on a magnitude of the deviations.

In one embodiment, the processing logic is operable to provide an indication of identified FRET interactions.

In one embodiment, the processing logic is operable to provide the indication of identified FRET interactions spatially correlated with an image of the sample.

In one embodiment, each region is imaged by a different detector element.

In one embodiment, each region is imaged by the same detector element located at different positions.

In one embodiment, the neighbouring regions comprise adjacent pixels of the image.

In one embodiment, the neighbouring regions comprise an array of adjacent pixels of the image.

According to a fifth aspect, there is provided an imaging method, comprising: detecting photon emissions generated in response to optical stimulation by a sample to be imaged; and identifying fluorescing molecules within the sample by identifying differences in detected emission decay rates occurring within different regions of the sample.

In one embodiment, the method comprises detecting photon emissions generated over a detection time period in response to the optical stimulation.

In one embodiment, the fluorescing molecule comprise molecules undergoing FRET interactions.

In one embodiment, the method comprises providing an indication of FRET efficiency based on a magnitude of the differences in the detected photon emission decay rates within the different regions of the sample.

In one embodiment, the method comprises identifying differences in the detected photon emission decay rates occurring over each detection time period within the different regions of the sample.

In one embodiment, the method comprises identifying differences in the detected photon emission decay rates by comparing integrated emissions detected over each detection time period in the different regions.

In one embodiment, the method comprises identifying differences in the detected photon emission decay rates by determining a change in a centre of mass of integrated emissions detected within each detection time period across the different regions.

In one embodiment, the method comprises providing an indication of FRET efficiency based on a rate of change of the centre of mass.

In one embodiment, the different regions are neighbouring regions.

In one embodiment, the neighbouring regions are at least partially overlapping regions

In one embodiment, the method comprises identifying differences in the detected photon emission decay rates by utilising integrated emissions detected over each detection time period in each at least partially overlapping region to provide a spatially-correlated intensity point spread function.

In one embodiment, the method comprises identifying FRET interactions from an asymmetry in the spatially-correlated intensity point spread function.

In one embodiment, the method comprises identifying differences in the detected photon emission decay rates by utilising integrated emissions detected over subsets of each detection time period in each at least partially overlapping region to provide at least one spatially-correlated intensity point spread function.

In one embodiment, the method comprises spatially locating a source of the emissions by fitting one or more emission curves to each spatially-correlated intensity point spread function, a centre of mass of each fitted emission curve spatially locating a source of the emissions.

In one embodiment, the method comprises identifying FRET interactions from a change in a centre of mass of each spatially-correlated intensity point spread function over each subset of the detection time period.

In one embodiment, the method comprises spatially locating a source of FRET interactions from the change in the centre of mass.

In one embodiment, the method comprises spatially locating the source of FRET interactions by extrapolating the change in the centre of mass.

In one embodiment, each subset of the detection time period comprises a different subset of the detection time period.

In one embodiment, each subset of the detection time period comprises overlapping subsets of the detection time period.

In one embodiment, each subset of the time period iteratively excludes one of later and earlier detected photon emission within the detection time period.

In one embodiment, the method comprises identifying differences in the detected photon emission decay rates by identifying deviations from a predefined decay rate over each detection time period in each region.

In one embodiment, the method comprises providing an indication of FRET efficiency based on a magnitude of the deviations.

In one embodiment, the method comprises providing an indication of identified FRET interactions.

In one embodiment, the method comprises providing the indication of identified FRET interactions spatially correlated with an image of the sample.

In one embodiment, the method comprises imaging each region with a different detector element of a detector array.

In one embodiment, the method comprises imaging each region with the same detector element located at different positions.

In one embodiment, the neighbouring regions comprise adjacent pixels of the image.

In one embodiment, the neighbouring regions comprise an array of adjacent pixels of the image.

According to a sixth aspect, there is provided a computer program product operable, when executed on a computer to control an imaging apparatus to perform the method of the fifth aspect.

Further particular and preferred aspects are set out in the accompanying independent and dependent claims. Features of the dependent claims may be combined with features of the independent claims as appropriate, and in combinations other than those explicitly set out in the claims. In particular, features of the first and fourth aspect and features of the second and fifth aspects may be combined.

Where an apparatus feature is described as being operable to provide a function, it will be appreciated that this includes an apparatus feature which provides that function or which is adapted or configured to provide that function.

### BRIEF DESCRIPTION OF THE DRAWINGS

Embodiments of the present invention will now be described further, with reference to the accompanying drawings, in which:

FIGS. 1A and 1B illustrate imaging apparatus according to embodiments;

FIG. 2 illustrates the operation of the imaging apparatus;

FIG. 3 illustrates a raster scan pattern;

FIGS. 4A to 4E illustrates rotated scan patterns according to embodiments;

FIG. 5 illustrates an imaging apparatus according to one embodiment;

FIG. 6a illustrates a fluorescence decay curve;

FIG. 6b illustrates an integrated fluorescence decay curve for different sub-periods within a detection period;

FIG. 7 illustrates the main processing steps performed by the imaging apparatus;

FIG. 8 illustrates a fluorescence intensity plot according to one embodiment;

FIGS. 9A to 9C illustrate how movement of a location of a centre of mass provides an indication of interacting and non-interacting fluorophores; and

FIG. 10 illustrates an imaging apparatus according to one embodiment.

### DESCRIPTION OF THE EMBODIMENTS

Before discussing embodiments in more detail, first an overview will be provided. Embodiments provide an arrangement which provides for high resolution, fast and simple imaging of a sample. An optical assembly generates an array of, typically photon, beams which are scanned with respect to a sample along a single scanning axis. That is to say that each beam is scanned with respect to the sample in a single, continuous scanning line rather than scanning a plurality of scanning lines each offset from the other as found in raster scanning approaches. A detector having an array of detector elements detects emissions, typically photons, generated by the sample in response to the array of beams. This enables an area of the sample to be imaged continuously as the array of photon beams move with respect to that sample without needing to displace each beam to a new scanning line as found in raster scanning approaches. By tilting the orientation of the array of beams with respect to the scanning axis, the field of view of the array of beams can be varied to vary the imaging area as the beams move with respect to the sample. The orientation of the array of beams can be varied to provide for beams which do or do not scan the same portion of the sample. Orientating the beam array such that none of the beams scan the same portion of the sample maximises the spatial area of sample being imaged. Arranging for beams to scan the same portion of a sample reduces spatial coverage during imaging, but can help to compensate for variations between different detector elements. Interpolation can be used to interpolate between the scan lines scanned by the beams to reconstruct those portions of the sample which may not be scanned. This approach enables the array of beams to be swept over the sample very quickly since the beams need only scan along a single axis, typically in a single sweep with respect to the sample. This enables the sample to be imaged much more quickly and with reduced complexity compared to existing raster scanning approaches. Also, the speed at which the beams scan with respect to the sample can be determined in order to vary the number of emissions used to generate each pixel in order to prevent image blurring.

Embodiments are particular suited to fluorescence lifetime imaging microscopy (FLIM) which is a well-established method for high resolution imaging of the functional spatio-temporal dynamics in situ using a variety of techniques, Förster resonance energy transfer (FRET) being by far the most extensively studied for protein-protein homo- and hetero-dimer interactions. For intermolecular FRET, a key benefit of performing donor FLIM (when compared to intensity based ratiometric techniques), is that fluorescence-lifetime measurements of donor emission are independent of acceptor concentration (assuming there is excess acceptor) and is thus suited to imaging studies in intact cells. Confocal and multiphoton microscopy confers additional advantages in terms of three dimensional sectioning and, in the case of multiphoton microscopy, enhanced depth penetration for in vivo imaging. However, the data acquisition rate for FLIM is a significant limitation in current implementations of laser scanning microscopy.

For high precision FLIM, time-correlated single photon counting (TCSPC) is unparalleled in its measurement accuracy. In terms of imaging speed, TCSPC is fundamentally limited with respect to photon counting rate, since the stochastic nature of the emission process requires that the detection rate is much less than one photon per excitation event to prevent inaccuracies in lifetime determination due to pulse pile-up. Even with multi-hit time-to-amplitude converters there are fundamental limits to the number of photons per unit volume per unit time that can be extracted (dependent on concentration, fluorophore photophysics etc). Consequently, typical acquisition times for existing laser scanning FLIM are in the order of minutes, whereas many dynamic biological events occur on significantly faster timescales. Whilst resonant scanners can achieve such rates for laser scanning microscopy, often signal-to-noise is limiting due to both phototoxicity and detection efficiency. In order to overcome these limitations, parallel signal acquisition using arrays of laser beams with either photomultiplier arrays or time-gated camera detection systems may be employed. Accurate determination of fluorescence lifetime with large numbers of channels in such a parallel manner may be limited either due to cross-talk in multi-anode photomultipliers or subject to systematic error due to measurement methodology.

### Imaging Apparatus—Mechanical/Optical Scanning

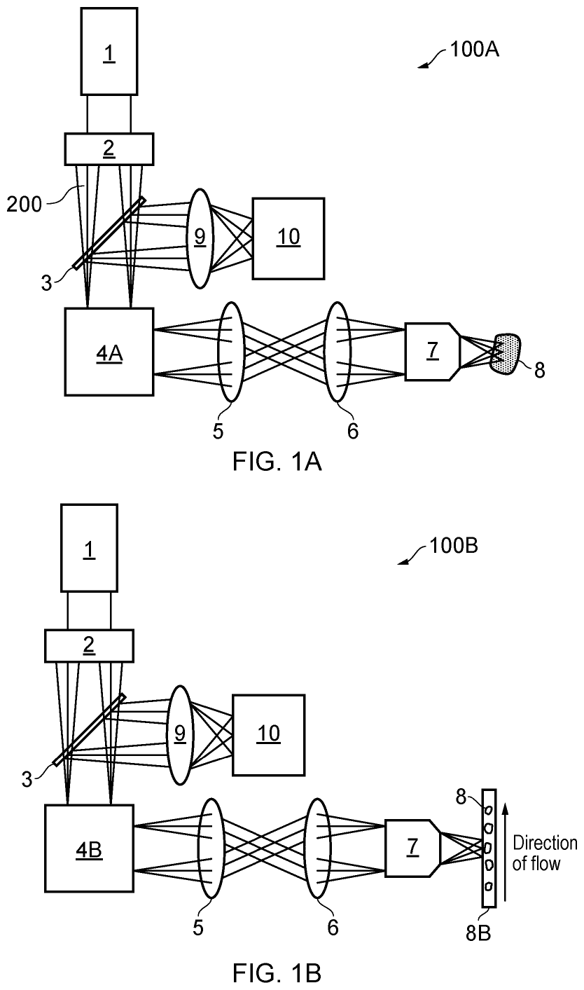

FIG. 1A illustrates an imaging apparatus, generally 100A according to one embodiment. A pulsed light source 1 (such as a laser) is optically coupled with a multi-focal generation element 2. The multi-focal generation element 2 is optically coupled with a dichroic filter 3. The dichroic filter 3 is optically coupled with a single axis scanning element 4A and with reimaging optics 9. The reimaging optics 9 is optically coupled with a detector array 10. The single axis scanning element 4A is optically coupled with a tube lens 5, which is optically coupled with a scan lens 6, which is optically coupled with an objective 7. The objective 7 is optically coupled with a sample 8. Hence, this arrangement provides a confocal imaging arrangement.

### Imaging Apparatus—Continuous Flow Scanning

FIG. 1B illustrates an imaging apparatus, generally 100B according to one embodiment. A pulsed light source 1 (such as a laser) is optically coupled with a multi-focal generation element 2. The multi-focal generation element 2 is optically coupled with a dichroic filter 3. The dichroic filter 3 is optically coupled with a beam steering optics 4B and with reimaging optics 9. The reimaging optics 9 is optically coupled with a detector array 10. The beam steering optics 4B is optically coupled with a tube lens 5, which is optically coupled with a scan lens 6, which is optically coupled with an objective 7. The objective 7 is optically coupled with flow cell 8B through which a sample 8 flows. Hence, this arrangement provides a confocal imaging arrangement.

### Imaging Operation

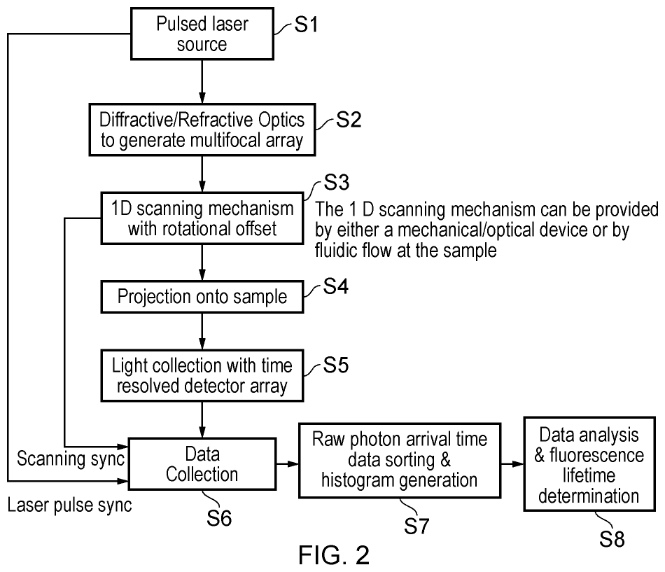

The operation of the imaging apparatus 100A, 100B is described with reference to FIG. 2. At step S1, the pulsed light source 1 generates photons, at a pulse repetition rate commensurate with the fluorophore lifetime (typically, for standard embodiments, this is in the range 1-80 MHz).

At step S2, the emitted photons pass through the multi-focal generation element 2 which generates an array of beamlets 200. In these embodiments, the beamlets 200 are arranged in an N×N square array, however, it will be appreciated that other rectangular and non-rectangular arrays are possible such as, for example, a hexagonal close packed arrangement.

At step S3, the array of beamlets 200 are scanned with respect to the sample 8, typically with a selected rotational offset. In the embodiment shown in FIG. 1A, such scanning is performed by the single axis scanning element 4A. In the embodiment shown in FIG. 1B, the scanning is performed due to the movement of the sample 8 within the flow cell 8B with the rotation being achieved by rotation of the flow cell 8A and/or by a rotation introduced by the beam steering optics 4B.

At step S4, the rotated array of beamlets 200 illuminate the sample 8.

At step S5, the emissions from the sample 8A pass back through the objective 7, scan lens 6, tube lens 5, single axis scanning element 4A/beam steering optic 4B, to the dichroic filter 3 where they then pass to the reimaging optics 9 and onto the detector array 10.

At step S6, data relating to received emissions is collected. Typically, the imaging apparatus 100A, 100B will be used for detecting Förster resonance energy chancer (FRET) interactions. Accordingly, for pulsed photon emissions from the pulsed light source 1 at a rate of 80 MHz, it is expected that emissions from the sample 8 will occur at a rate of less than 0.8 MHz.

At step S7, each detector element in the detector array 10 is time-gated with the pulsed light source 1 and cumulatively measures the time interval between 1 photon illuminating the sample 8A and an emission from the sample occurring. Those time intervals are summed using a histogram.

At step S8, those histograms are analysed to determine a decay curve, from which both fluorescence lifetimes and, by interpretation, FRET interactions are determined.

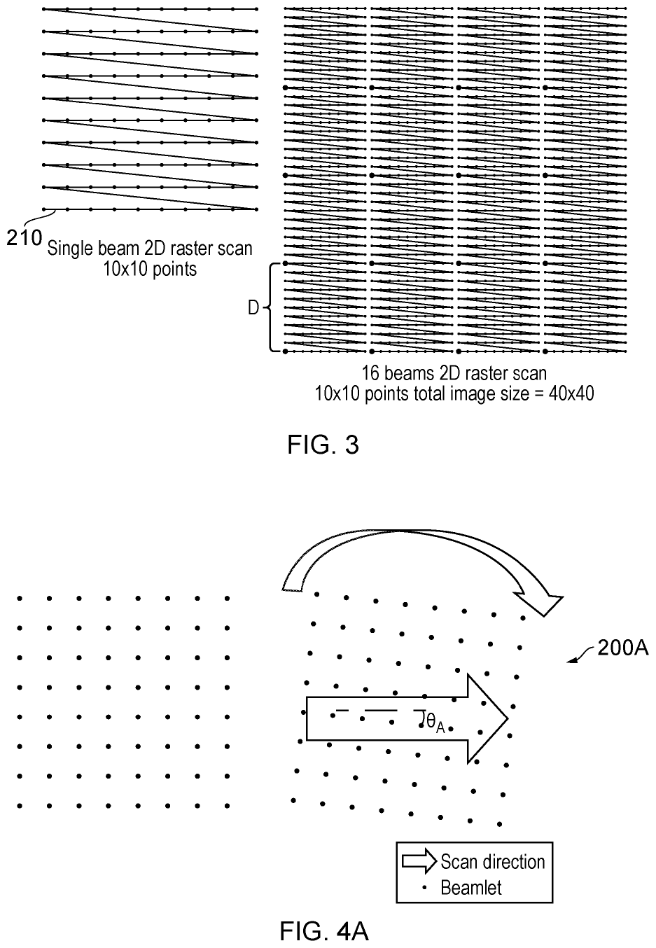

Detector Array As shown in FIG. 3, in order to prevent various forms of crosstalk, such single photon detector arrays typically have a detector element 210 diameter of around 6 microns, but a spacing D between detectors of around 50 microns. Accordingly, in order to image the sample on to the detector array, it will be necessary to perform a raster scan in the sample plane, as illustrated in FIG. 3. Such scanning requires each beamlet associated with a detector element 210 to be scanned in two axes across parallel scan lines, a number of times for each scan line. In this example, each single scan line collects data at 10 different points on 10 parallel scan lines, which (for a 4×4 detector array) gives an image with data collected at 40×40 points.

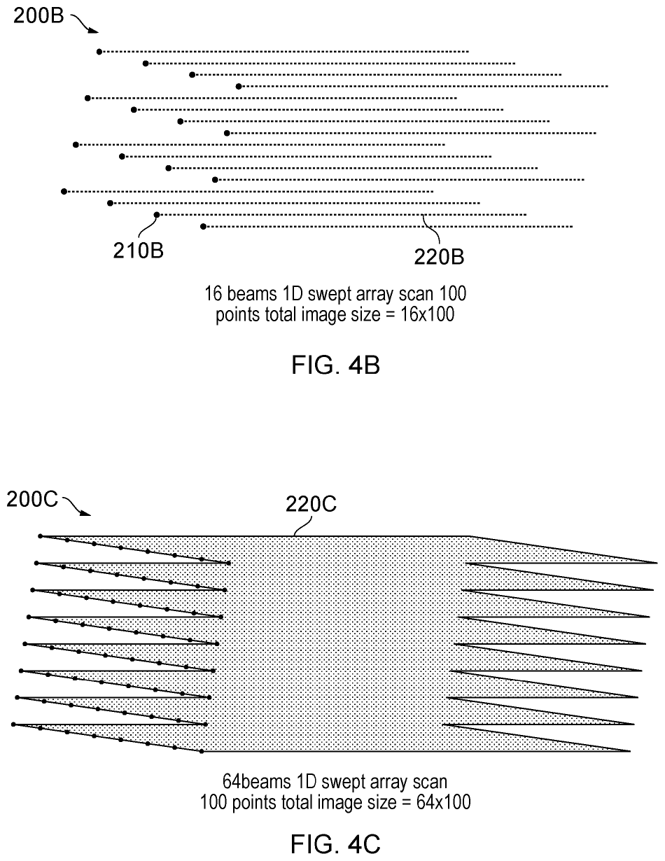

However, as illustrated in FIG. 4A, embodiments instead rotate the array of beamlets 200A by an angle θ_{A} with respect to the direction of scan.

As shown in FIG. 4B, by rotating the array of beamlets 200B with respect to the direction of scan, each beamlet 210B can trace its own scan line 220B to image the sample. In this example each single scan line collects data at 100 different points, which (for this 4×4 detector array) gives an image with data collected at 16×100 points.

Looking now at FIG. 4C, in this arrangement, the array of beamlets 200C is orientated to provide a uniform distribution of scan lines 220C over the sample. In this example each single scan line collects data at 100 different points, which (for this 8×8 detector array) gives an image with data collected at 64×100 points.

As shown in FIG. 4D, the degree of rotation of the array of beamlets 200D can be increased further so that some beams 210D scan the same scan line 220D over the same location of the sample 8, albeit at different times. This means that the same portion of the sample 8 is illuminated twice and the emissions from that portion of the sample 8 detected with different detector elements in the detector array 10. This allows for detector elements which fail to function optimally within the detector array 10 to be compensated for by either disregarding or correcting their data. Also, this enables different temporal images to be constructed.

As shown in FIG. 4E, the array of beamlets 200E can be rotated further so that up to four beamlets 210E scan the same scan line 220E over the same portion of the sample and those emissions are detected by four different detector elements.

Although in this embodiment a confocal arrangement is used, it will be appreciated that different optical arrangements are possible which may use the same scanning technique. The speed of the scan with respect to the sample can be used to reduce blurring in any images produced. That speed can be determined in the arrangement shown in FIG. 1A from the speed of the single axis scanning element 4. In the embodiment shown in FIG. 1B, the speed of the sample can be determined from the speed at which features of the sample (or even from beads introduced into the flow cell 8) put together with the sample pass through the image.

Embodiments utilise multi-beam excitation and detection capabilities for use in confocal and multiphoton fluorescence imaging which can be attached to any existing microscope. Beamlet generation will be provided using a diffractive optical element (DOE). A DOE specifically designed for a particular wavelength will have higher diffraction efficiency with greater uniformity between beamlets. Beam scanning is performed utilizing a single axis scanning mechanism and generated fluorescent light is then descanned and projected via a set of appropriate filters onto a detector array.

Embodiments typically utilise a DOE designed for the application; alignment and calibration procedures to efficiently align beamlets generated by the DOE onto the detector array; confocality of the microscope is achieved due to the specific size of the active region of the detector and the projection objective used. Zoom optics can be incorporated to offer continuous variation and to modify confocality; scanning is performed using a single axis scanner with an angular offset on the 2-D beam array—the beamlets remain stationary with respect to the detector during the scan due to descanning. For an arbitrary array of beams (with dimension n×n), with evenly spaced beams, by simple trigonometry:

$$∅ = {tan}^{- 1} \frac{1}{n}$$

For 32×32 beamlets, an active area d=6 microns and a centre to centre active area distance D=50 microns, this would equate to an angular offset to the axis parallel to the beamlet array axis of approximately 1.7975 deg. (to 2sf) and 1024 beamlets; angle modification can be performed via rotating the image formed or by rotating the angular position of the mirror with respect to the optical axis and this will serve a number of functions: (a) to allow crossover of multiple tracks; (b) measure temporal delay; since only one axis is involved and the scan only needs to be scanned across the scan range once per image acquisition; FLIM imaging can be performed at acquisition rates faster than any other single beam FLIM based system and with comparable temporal precision; the system is developed primarily for high content pathological screening applications to but could also be used in general microscopy.

Embodiments provide the following advantages: Ease of use—embodiments can be attached onto any existing microscope system; Speed—acquisition rates faster than any other beam scanning FLIM based system; Accuracy—temporal measurement accuracy provided by TCSPC to measure lifetime; Cost Effective—this technique has the potential to be much less expensive than existing FLIM based systems.

Embodiments are essentially a paradigm shift over existing fluorescence lifetime imaging tools and provides the user with the ability to monitor protein-protein interactions in a high content screening environment

The set-up provides a platform for future improvements in speed and signal-to-noise by increasing the number of beams or using smaller area SPADs. Such advances have the potential to transform time-resolved multiphoton imaging applications in a range of biological systems. Now we can utilise unparalleled temporal measurement accuracy provided by TCSPC to measure lifetime with high frame rates to image complex live cell interactions dynamically.

Embodiments would be primarily used to image FRET interactions for high speed pathological screening but can be used either in in-vivo, in-vitro and ex-vivo imaging situations. This includes protein-protein homo- or hetero-dimer interactions and with FRET biosensors. Embodiments can also be used with fluorescence lifetime probes to monitor localised environment variations. Embodiments provide a self-contained module could be attached onto a number of existing microscopy systems such as confocal, multiphoton, endoscopic, high content screening and flow imaging.

Embodiments seek to bridge the gap between flow cytometry and fluorescence lifetime imaging microscopy. Flow cytometry is a technique for unbiased screening of whole-cell fluorescence intensity (and scattering) signature of cells flowing in suspension. Fluorescence lifetime imaging microscopy (FLIM) creates images of immobilized cells (in tissue or coverslip) with contrast offered by the fluorescence lifetime of fluorescent constituents of the cells. Imaging flow cytometry combines the advantages of flow cytometry and fluorescence microscopy to measure the sub-cellular spatial distribution of fluorescent constituents of populations of flowing cells. Embodiments seek to advance imaging flow cytometry to enable generation of confocal images with fluorescence intensity as well as lifetime contrasts at high-throughput unbiased in the selection of analysed cell.

The requirement for imaging flow cytometers comes from the advantage of high throughput of the flow cytometer and the morphological and spatial information about analysed cells contained in the images. This has led to commercial developments in the recent past. None of the known approaches offer the capacity to measure fluorescence lifetime by the method of time correlated single-photon counting (TCSPC). Furthermore, none the known instruments allows confocal imaging, which would provide optical sectioning and thus high image contrast without interference from out-of-focus fluorescence. Embodiments perform fluorescence lifetime imaging by performing multibeam confocal time-correlated single photon counting at n points in the sample concurrently. The cell passes through the imaging volume flowing in a direction tilted at an angle in respect to the measurement lattice of n points. Its cross-sectional confocal image is computationally reconstructed with fluorescence intensity and fluorescence contrast at one or more combinations of excitation and emission wavelengths. The sectioning capability of the confocal imaging yields flow cytometry images of unprecedented crispness and contrast. At the same time, fluorescence lifetime images offer unprecedented resolution and sensitivity due to the superior qualities of TCSPC for measurement of fluorescence lifetime. Furthermore, the effective sub-nanosecond time gate for photon detection offers reconstruction of images inherently void of any motion blur, a problem for which special techniques need to be employed in the aforementioned known techniques.

Embodiments provide a fusion of techniques that combined together enable the advancement, in particular: application of single-photon avalanche photodiode detectors; and fluorescence lifetime flow cytometry developments.

Imaging flow cytometry requires solving the inherent problem of avoiding motion induced blur while retaining sufficiently long integration time to ensure required image contrast. In embodiments, TCSPC time stamps of each photon (sub-nanosecond time resolution) together with the known position on the corresponding SPAD position on the array allow assigning the photon to accurate position within the sample without any motion blur. Reconstructing non-distorted image of flowing particles in any image flow cytometer requires the knowledge of the particle speed. The image-reconstruction described above has the same requirement.

In embodiments, a cross-correlation between images obtained at a sliding time window is used to infer the actual particle speed for each passing particle.

Image contrast in image flow cytometry depends on a number of factors, including the interference of out-of-focus blur.

In embodiments, the use of confocal detection on an array of SPADs will effectively remove majority of out-of-focus light and thus yield sectioned images, comparable to those from a spinning disc microscope.

Existing implementations of imaging flow cytometers measure fluorescence intensity only.

In embodiments, TCSPC time-stamps allow direct calculation of fluorescence lifetime contrast in the imaging flow cytometer images.

Embodiments deliver a benefit to existing techniques. Embodiments produce motion blue free images without the need for fiduciary particles or any restriction on the integration time. This simplifies the instrumentation design as well as experimental procedure.

Embodiments deliver sectioned images of the particles. Sectioned images can be produced by various types of microscopes but not by known imaging flow cytometers. Sectioned images provide increased contrast allowing for higher clarity of images of the studies particles (cells). The benefit can be compared to that of a confocal, spinning disc of selective plane illumination microscopes compared with a wide-field microscope.

Embodiments deliver directly-measured fluorescence lifetime images of flowing particles without the need for lifetime calibration.

Embodiments provide a form of a stand-alone instrument to be used in unbiased cell screening. Imaging flow cytometry is a technique positioned between conventional flow cytometry and microscopy. It brings together the advantages of both, the unbiased screening of large particle (cell) populations with the image representation of internal fluorophore distribution. Furthermore, it is particularly well suited for studying suspension cells. By adding the image sectioning capability and fluorescence lifetime readout, extra information from the analysed particles (cells) can be gained.

Before discussing further embodiments in any more detail, first another overview will be provided. Embodiments provide an imaging apparatus for detecting functional interactions within a sample. Typically, those functional interactions are identifiable due to molecules within a sample exhibiting different fluorescent lifetimes. Such different fluorescent lifetimes may occur due to physical, chemical or biological differences in the sample that is being imaged. Accordingly, a sample is provided and is imaged using the imaging apparatus. Typically, the imaging apparatus utilises a single, pulsed beam which scans over the sample to be imaged. Portions (typically molecules) within the sample respond to illumination by the beam and perform emissions in response. Those emissions are typically detected by a single detector. However, it will be appreciated that the sample may also be imaged using multiple beams and multiple detectors, in the manner to that described in the embodiments above. In any event, the pulsed light source illuminates the region of the sample in a pulsed manner for an illumination period of time. The detector measures the emissions from that region over a detection period in response to the illumination and records the time between each pulse being sent and an emission (which typically comprises a single photon) being received. It will be appreciated that the size of the illumination and detection areas and the length of the illumination and detection periods can be varied depending on requirements. Typically, the detection period will be longer than the illumination period. It is envisaged that the part of the sample (such as one or more molecules) performing the emissions in response to the illumination is unresolvable by the detector element which is diffraction-limited. The imaging of different regions of the sample is performed and their emissions are also recorded. Preferably, those different regions are neighbouring or adjacent and, more preferably, the different regions at least partially overlap. Once the sample has been imaged and the emissions have been recorded, then that data is processed in order to identify whether any interactions are occurring. By identifying differences in the detected emissions decay rates in different regions, it is possible to determine that fluorescing molecules may be experiencing different conditions or interactions. For example, differences in fluorescence lifetimes may indicate that identical molecules are experiencing different physical, chemical or biological conditions within the sample. One such condition is the occurrence of FRET due to coupling between one molecule and a neighbouring molecule. The processing typically also examines the nature of those fluorescence lifetimes in order to provide for super-resolution and identify the spatial location of a portion of the sample (for example, a molecule) within the diffraction-limited area of the detector. One technique for super-resolving the positions of these portions of the sample is to provide a spatially-collated intensity distribution of emissions over a number of overlapping regions and utilise changes in the centre of mass of that distribution to spatially resolve the position of a fluorescing molecule. That is to say that for a typical detector area of greater than around 250 microns, it is possible to resolve the position of a 10-20 nanometre portion of the sample. This technique is particularly suited to FRET interactions since the centre of mass of such a function which encompasses FRET interacting and non-interacting portions will tend to move from the interacting to the non-interacting portion over a time-period commensurate with the fluorescence lifetime.

### Imaging Apparatus

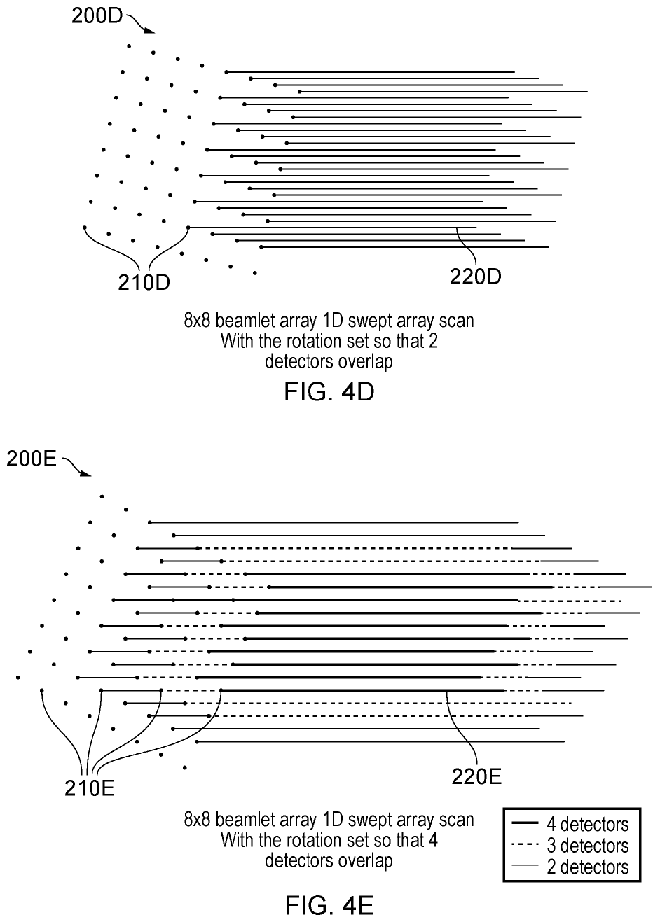

FIG. 5 illustrates an imaging apparatus, generally 3oo according to one embodiment. A pulsed light source 330 is optically coupled with a dichroic beam splitter 340. The dichroic beam splitter 340 is optically coupled with an objective lens 350 and a detector 360. The objective lens 350 is optically coupled with a sample 370. A computer 380 is coupled with a detector 360. In this embodiment, a single, pulsed beam is provided by the light source 330 and used to illuminate a region of the sample 370 via the dichroic beam splitter 340 and the objective lens 350. The light source 330 is pulsed, typically at a pulse frequency such as 80 megahertz (although it will be appreciated that other frequencies may be used), in order to cause fluorescence in molecules within the portion of the sample 370 being illuminated in a similar manner to the embodiment mentioned above. It will be appreciated that in other embodiments an additional low-intensity beam may be used to excite a small number of molecules. The operation of the light source 330 to achieve fluorescence activation or emission of only a small number of molecules in the region being illuminated is well known in the art.

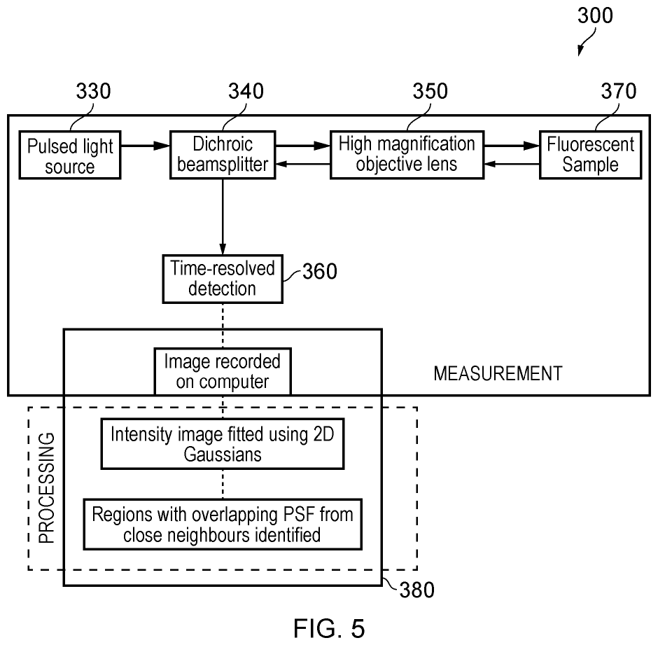

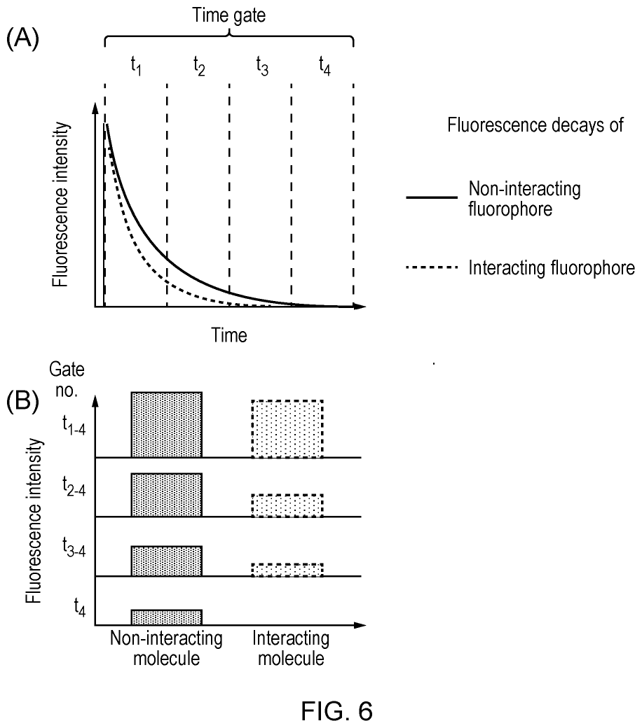

The time between each photon being emitted by the light source 330 and an emission from the sample 370 (which travels back through the objective lens 350 to diachronic beam splitter 340 and on to the detector 360) is recorded for many photon emissions from the light source 330. That timing information can be compiled into a histogram which provides a fluorescence decay curve, as illustrated in FIG. 6a. It will be appreciated that the sample or measuring rate of the detector 360 needs to be faster than the decay rate being observed. In this example, the detection period starts with period t1 and finishes with period t4. As can be seen, more emissions occur in period t1 than in t4.

As can also be seen in FIG. 6a, different fluorophores exhibit different decay rates. For those fluorophores which may be affected by FRET interactions, the decay rate changes depending on the extent or efficiency of the FRET interactions. For example, a fluorophore which does not have any FRET interaction decays more slowly than one which is undergoing a FRET interaction, as illustrated in FIG. 6a. Other fluorophores exhibit similar properties under other physical, chemical and biological conditions. By examining differences in these decay rates, it is possible to determine the presence of non-interacting and interacting fluorophores.

Looking now at FIG. 6b, it can be seen that when the area under the curve is integrated for different time periods, the integrated fluorescence intensity during those periods varies. For example, when summing across the complete detection period t1-4, similar fluorescence intensity is exhibited, although less fluorescence intensity is exhibited for the interacting fluorophore across the complete detection period t1-4 and this difference can be identified. When looking at subsets of the total detection period variation occurs. For example, it can be seen that in just time period t4, no fluorescence intensity from the interacting fluorophore is measured and the fluorescence intensity comes only from the non-interacting fluorophore, although it can be seen that less fluorescence intensity is exhibited for the interacting fluorophore within each time period and this difference can be identified. This observation can be used to determine the presence of non-interacting and interacting fluorophores within the region of the sample being illuminated.

Furthermore, by imaging different parts of the sample and then spatially correlating the fluorescence intensities measured over the detection period at each of those different locations, it is possible to spatially locate in a super-resolution manner interacting and non-interacting fluorophores since the centre of mass of the spatially correlated intensity function will shift towards the location of the non-interacting fluorophore for the later parts of the detection time, as will be explained in more detail below.

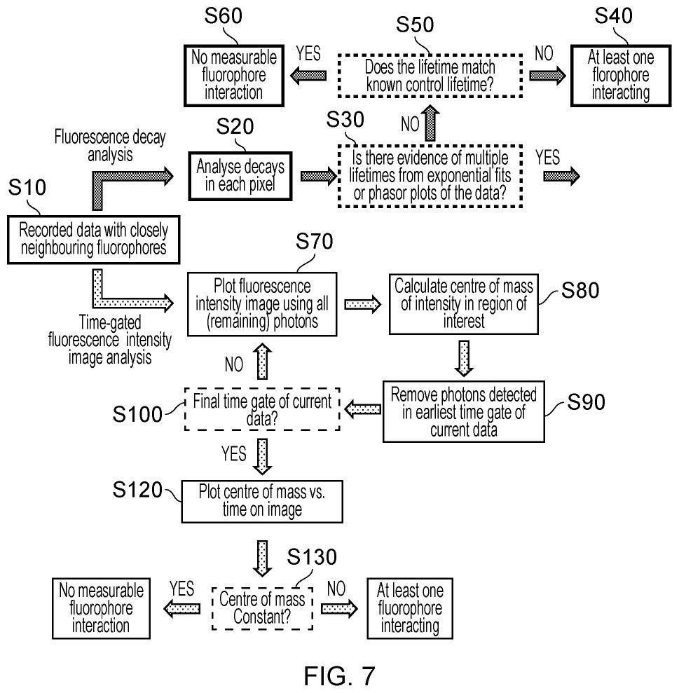

FIG. 7 illustrates the main processing steps performed by computer 380 following the illumination of the sample 370 at different locations and the recording of the emissions over the detection period at each illumination position on the sample 370.

Step S10 commences with the emission data for a plurality of sample positions having been recorded by the computer 380. Should fluorescence decay analysis be desired then processing proceeds to step S20. Should fluorescence intensity image analysis be desired then processing proceeds to step S70.

At step S20, the collected emissions in each pixel position of the sample are plotted as a decay curve, such as that illustrated in FIG. 6a and processing proceeds to step S30.

At step S30, those decays curves are analysed to see whether there is evidence of multiple lifetimes. It can be inferred that there are multiple lifetimes if the decay curve measured at that location fails to fit standard decay curves or standard analysis techniques preconfigured within the computer 380. If the decay curves fail to fit the standard decay curves or standard analysis techniques, then processing proceeds to step S40 where an indication is provided that at least one fluorophore is reacting. If there is no evidence that multiple lifetimes are present then processing proceeds to step S50.

At step S50, an assessment is made of whether the decay lifetime matches a known control decay lifetime. If the decay lifetimes do not match, then processing proceeds to Step S40 where an indication is provided that at least one fluorophore is interacting. If the lifetime does match a known control lifetime then processing proceeds to S60.

At step S60, an indication is provided that there is no measureable fluorophore interaction.

Hence, it can be seen that through decay curve analysis and decay curve fitting it is possible to provide an indication of the presence or not of interacting or non-interacting fluorophores in each pixel location.

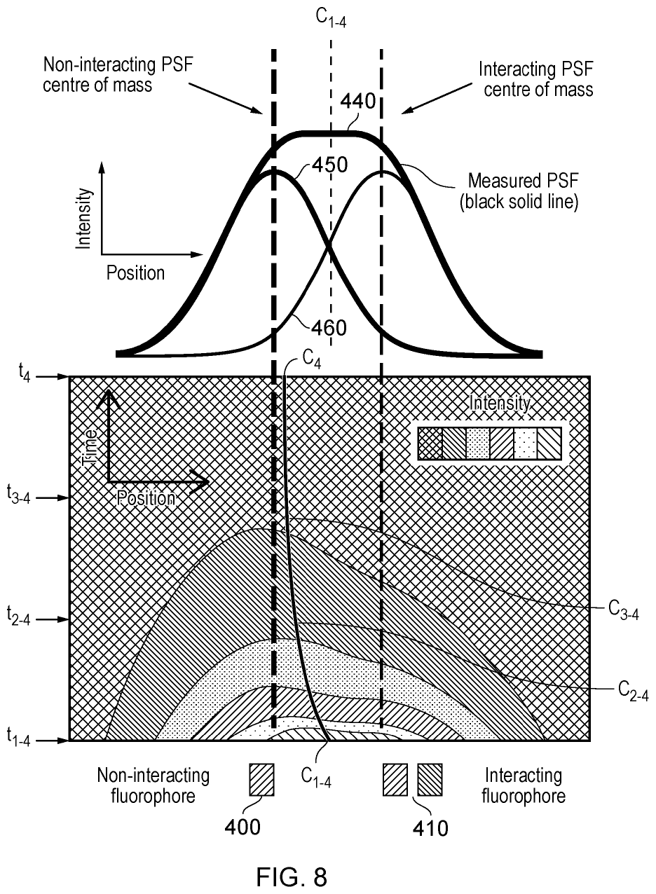

At step S70, data collected when imaging the sample 370 in the vicinity of the emissions is collated. For example, the data relating to emissions from overlapping portions of the sample 370 in the vicinity of the emissions are identified and a fluorescence intensity plot is generated, as illustrated in FIG. 8. In this example, there is one non-interacting fluorophore 400 and an interacting fluorophore 410. In this example, the diffraction limited area of the point spread function is approximately 250 nanometres and the size of the fluorophore is less than approximately 5 nanometres. Also in this example, the sample 370 in the vicinity of the fluorophores 400, 410 was illuminated a number of times by the pulse light source 330 and the emissions detected over the detection period by the detector 360 for each position. For example, the fluorophores 400, 410 may be illuminated four times by the light source 330 illuminating at different, overlapping positions. A fluorescence intensity distribution is then created by spatially correlating the measurements made by the detector 360 for each of the four positions. As can be seen, the correlated measured point spread function 440 is shown, as is the component 450 attributable to the non-interacting fluorophore 400 and the component 460 attributable to the interacting fluorophore 410.

At step S80, the centre of mass C1-4 of the correlated measured point spread function 440 is determined.

At step S90, the photons collected during the earliest time period is removed (in this case time period t1).

As step S100 a determination of whether data remains only for the latest time period is made. In this case that is not true since the data for each of time period t2, t3 & t4 remains and so processing proceeds to step S70 where the correlated measured point spread function 440 is re-plotted with the remaining data and at step S80, the new centre of mass C2-4 is determined.

This process continues to determine centre of mass C3-4 and C4 until at step S100 only the emission data relating to time period t4 remains and processing proceeds to step S120 where the centre of mass C verses time is plotted on the image, as shown in FIG. 8 and processing proceeds to step S130.

Although in this embodiment, earlier time periods are removed iteratively, it will be appreciated that the reverse can be used where later time periods are removed iteratively until only the earliest time period remains.

At step S130, a determination is made as to whether or not the location of the centre of mass C remains constant.

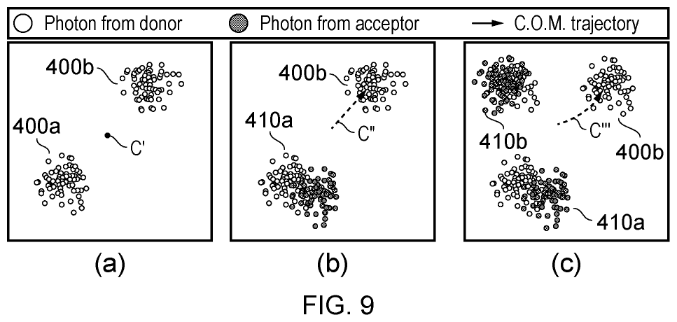

Considering the example in FIG. 9a, if the location of the centre of the mass C′ remains constant, then a determination can be made that no fluorophore interaction was present. The location of the non-interacting fluorophores 400a, 400b can then be spatially determined from the location of fitted curves which can be derived from the correlated measured point spread function.

Considering the example in FIG. 9b should the centre of mass C″ move, then it can be determined that the centre of mass C″ moves towards the location of a non-interacting fluorophore 400b and away from an interacting fluorophore 410a. Again, the location of both of those fluorophores can be determined by fitting curves to the correlated measured point spread function.

Considering the example in FIG. 9c, the centre of mass C′″ changes location non-linearly. According, it can be inferred that the centre of mass C′″ moves towards the non-interacting fluorophores 400b and away from two interacting 410a, 410b. Again, the location of those fluorophores can be determined by matching curves to the correlated measured point spread function.

Embodiments provide a method which allows for functional super-resolution imaging whereby imaging resolution exceeds that prescribed by the Abbé criterion, using differences in the fluorescence lifetimes of neighbouring molecules to distinguish them. Following activation of a sparse subset of molecules in a field of view into an emissive state (as in PALM imaging), the fluorescence is measured using time-resolved detection (on a sub-nanosecond timescale) following pulsed excitation such that intensity images can be generated at arbitrary time points along the nanosecond fluorescence decay transient of the fluorescence. In the case that there is a single molecule emitting, or there two or more molecules emitting simultaneously with exactly the same fluorescence lifetime, the centre of mass of the PSF in the intensity image will be invariant during the fluorescence decay. However, if there are two or more molecules present in the PSF with a total of two or more fluorescence lifetime components in the PSF, then the centre of mass of the PSF will vary on the timescale of the fluorescence decay. Thus, it is possible to distinguish and localize two molecules emitting simultaneously within a PSF.

A major benefit of embodiments is the possibility of determining whether there are highly localized variations in the local environment of a fluorophore using the fluorescence lifetime without the need to fit the time-resolved data to a model. The anticipated primary application of the method allows for functional super-resolution by identification of intermolecular interactions (such as in protein FRET biosensors) and protein-protein interactions between fluorescently labelled molecules by FRET. In this case, if there are two adjacent molecules within the emission PSF, and neither molecule is undergoing FRET, then the time-invariant centre of mass will have no trajectory. However, if one of the molecules is undergoing FRET, then the centre of mass of the emission PSF will vary in time, tending towards the molecule with the longest fluorescence lifetime (not undergoing FRET), and in the plane of the image the trajectory of the centre of mass will be linear. In the case of a sample where there are more than two fluorophores in the PSF and a distribution of FRET efficiencies between fluorophores then the time-evolving PSF will follow a nonlinear trajectory, still tending towards the position of the longest lifetime fluorophore. Thus, the presence of a FRET interaction, and also information regarding the number of molecules emitting simultaneously within the PSF is measurable from sub-nanosecond time-resolved data by simply observing the evolution of the intensity centre of mass of the PSF with no lifetime fitting. Thus, the presence of FRET is observable with no prior knowledge of the sample composition, and with detection in a single spectral window (that of the donor emission).

If one parameter, the donor lifetime in the absence of acceptor for a FRET pair, is known, then the FRET efficiency can also be quantified from the measurements by fitting the fluorescence decay and extracting the lifetime(s) of the donor molecules undergoing FRET. The fractional contributions of the lifetimes will further provide information regarding the number of molecules in the PSF.

The ability to reliably define the presence and position of more than one molecule emitting simultaneously in a PSF can reduce the number of acquisition frames necessary to generate a super-resolved image. In addition, the measurements probe function and provide additional information regarding protein-protein interactions in the sample.

Unique to embodiments is the calculation of the evolution of the fluorescence centre of mass on the timescale of the fluorescence decay. In order to localise two adjacent alike fluorophores in the case where one is undergoing a FRET interaction, only one parameter (the fluorescence lifetime of the non-interacting donor molecule) is required. Time-resolved fluorescence measurements have not been used in localisation microscopy for the purposes of determining whether a FRET interaction is occurring whilst also localising the position of the donor. Nor have measurements been made where a fluorescence lifetime component is an unknown, as it is in a FRET-FLIM experiment.

Embodiments provide a simple visual method, based only on the calculation of the centre of mass which involves no mathematical fitting of the fluorescence lifetime data, for identification of one, two, or more molecules within the PSF.

Embodiments provide for picosecond time resolution. To this end embodiments include a SPAD array. Background photons due to noise of light-scatter from the sample may be suppressed by TCSPC imaging by subtraction or incorporation into fitting algorithms. Background is often a limiting factor in obtaining high resolution localization microscopy images.

Advantages of embodiments include:

1) Dynamic super-resolution functional imaging of interactions, with identification of FRET without the necessity to fit the time-resolved data to a model, and using a single detection channel, with no chromatic aberrations.
2) Fluorescence lifetime measurements provide information on the local environment of the fluorophores on spatial scales below those of super-resolution imaging→structure-function information rather than just structural information.
3) More than one molecule in a PSF can be detected and localization achieved even if both are emitting simultaneously. This can potentially reduce the number of frames, and consequently the time necessary to acquire a super-resolution image.
4) FRET can be identified by monitoring the spatial trajectory of the PSF as a function of time.

Embodiments utilise a multibeam confocal fluorescence microscope which is capable of rapid fluorescence lifetime imaging, with a detector comprising a 32×32 array of single photon avalanche diodes (SPADs) with each SPAD capable of time-resolved detection with picosecond resolution. This will drastically reduce data acquisition times compared with standard single beam scanning FLIM systems, making overall data collection rates comparable to current widefield super-resolution (<1 s per frame) with the added advantage of the SPADs being high sensitivity detectors.

### Imaging Apparatus—Isotropic Arrangement

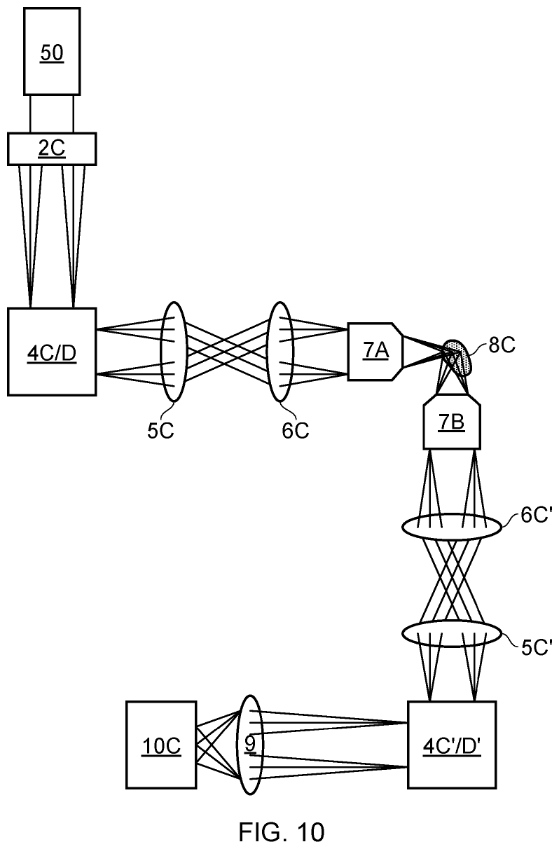

FIG. 10 illustrates an imaging apparatus, generally 100C according to one embodiment. A pulsed light source 50 (such as a laser) generates a light beam which is optically coupled to a diffractive optical element 2C (or similar such device) which generates an array of beam foci in 3 dimensions along the optical axis. The diffractive optical element 2C is then optically coupled with an X axis scanning element 4C. The X axis scanning element 4C is optically coupled with a Z axis scanning element 4D. The Z axis scanning element 4D is optically coupled with an excitation objective 7A via a scan lens 5C and a tube lens 6C. The excitation objective 7A is optically coupled with a sample 8C.

A detection objective 7B is optically coupled with the sample 8C. The detection objective 7B is optically coupled via a tube lens 6C′ and a scan lens 5C′ with a first descanning element 4C′ to remove lateral scan motion. The first descanning element 4C′ is optically coupled to a second descanning element 4D′ which corrects for de-focus (z-scanning). The second descanning element 4D′ is optically coupled to an imaging lens 9. The imaging lens 9 is optically coupled with a detector array 10C. Scanning elements 4C and 4D are electrically synchronized with descanning elements 4C′ and 4D′ to provide a stationary beam at the detector array 10C. This arrangement is bidirectional and therefore provides two orthogonal views of the sample 8C, which are combined by software to provide isotropic 3D digitally scanned light sheet time-domain fluorescence lifetime imaging (FLIM). This arrangement is an extension of the earlier embodiments for fluorescence lifetime imaging (SWARM—Swept Array Microscopy) and provides a digitally scanned light sheet mode of operation.

In general, the capabilities of the embodiments mentioned above provide for up to 40 Hz imaging using either photon counting or time-correlated single photon counting modes with diffraction limited imaging performance with up to 1024 parallel excitation and detection channels in either 2-photon or single photon excitation modality. However, this embodiment uses a Megaframe 32 camera (MF32, now available from Photon Force Ltd) as the detector array 10C with functionality to integrate real-time digital signal processing (S. Poland, A. Erdogan, N. Krstajic, J. Levitt, V. Devauges, R. Walker, D. Li, S. Ameer-Beg, and R. G. Henderson, (2016) New high-speed centre of mass method incorporating background subtraction for accurate determination of fluorescence lifetime, Opt. Express 24, 6899-6915.) to provide a selective plane microscope platform.

This embodiment allows diffraction limited imaging of a horizontal plane through the sample 8 perpendicular to the plane of the excitation objective 7a (lens) projecting a complex array of beams comprising individual Gaussian foci arranged in the horizontal plane to avoid cross-talk and synchronous detection via the orthogonal detection objective 7B (lens). Scanning can be achieved either through motion of the sample 8 or through synchronous scanning of the foci using high-speed galvos for both lateral and axial scanning. Scanning of a single plane requires only scanning in a single axis although arbitrary slice scanning may also be achieved using a combination of synchronised scanning elements. Acquisition of simultaneous adjacent planes is also possible. In the detection path, the MF32 is used as an array of pin-hole detectors to capture the emission with little or no cross-talk between the elements (due to significant physical separation between detectors). As such, this system has confocal apertures arranged in a Theta microscopy geometry (by E. H. K. Stelzer and S. Lindek, “Fundamental reduction of the observation volume in far-field light microscopy by detection orthogonal to the illumination axis: confocal theta microscopy,” Opt. Commun. 111, 536-547 (1994).). Whilst confocal apertures are not required in the 2-photon excitation case in a theta microscope the single view excitation/detection geometry leads to isotropic resolution through the combination of focused excitation and apertured detection. This allows extension to a bidirectional (i.e. reversed excitation/detection) methodology such that isotropic resolution is further improved. The platform offers an opportunity to provide interlaced 2-photon en face imaging modes if this was considered advantageous (i.e. without changing microscope platform—acquire both non-linear imaging and isotropic light-sheet). Whilst the system would see most utility in a 2-photon excitation modality, linear excitation is feasible albeit with slightly less utility due to out-of-plane excitation.

Although illustrative embodiments of the invention have been disclosed in detail herein, with reference to the accompanying drawings, it is understood that the invention is not limited to the precise embodiment and that various changes and modifications can be effected therein by one skilled in the art without departing from the scope of the invention as defined by the appended claims and their equivalents.

1. A time-resolved luminescence imaging apparatus, comprising:
an optical assembly operable to generate an array of beams;
a scanner operable to scan said array of beams with respect to a sample, along a single scanning axis; and
a detector assembly having an array of detector elements, adjacent detector elements being spaced apart by an inter-element gap, each detector element being operable to detect emissions generated by said sample in response to said array of beams.

2. The apparatus of claim 1, wherein a diameter of each detector element is less than said inter-element gap.

3. The apparatus of claim 1, wherein each detector element has a fill factor of less than 50%.

4. The apparatus of claim 1, wherein:
each detector element is operable to perform time-correlated single photon counting, and
each detector element comprises a single-photon avalanche diode.

5-6. (canceled)

7. The apparatus of claim 1, wherein said scanner is operable to scan said array of beams, each beam providing a scan line over said sample along said single scanning axis.

8. The apparatus of claim 1, wherein said array of beams comprise beams arranged in rows, extending along a beam row axis and in columns, extending along a beam column axis and said scanning axis is orientated between said beam row axis and said beam column axis.

9-16. (canceled)

16. The apparatus of claim 1, wherein said scanner comprises an optical scanner operable to direct said array of beams over said sample, along said scanning axis.

17. The apparatus of claim 1, wherein said scanner comprises a sample positioner operable to move said sample to direct said array of beams over said sample, along said scanning axis.

18. The apparatus of claim 17, wherein said sample positioner is operable to orientate a conduit, through which said sample is conveyed, along said scanning axis.

19. (canceled)

20. The apparatus of claim 1, further comprising processing logic operable to generate a sample image from detection data provided by each detection element in response to detected emissions.

21. The apparatus of claim 20, wherein said processing logic is operable to generate said sample image using a logic-provided indication of said orientation of said scanning axis.

22. The apparatus of claim 20, wherein said processing logic is operable to generate said sample image by interpolating said scan lines to generate unscanned portions of said sample image.

23. The apparatus of claim 20, wherein said processing logic is operable to compensate for detector element variation using overlapping scan lines when generating said sample image.

24. The apparatus of claim 20, wherein said processing logic is operable to disregard data generated by detector elements exhibiting greater than a selected variation.

25. The apparatus of claim 23, wherein said processing logic is operable to generate temporally-separated sample images using overlapping scan lines.

26. (canceled)

27. The apparatus of claim 20, wherein said processing logic is operable to determine a speed at which said array of beams scan over said sample and to determine said speed in response to at least one of an indication of a movement speed of said optical scanner and an indication of a sample speed determined from successive sample images.

28. The apparatus of claim 20, wherein said processing logic is operable to vary a number of detector emissions used to generate each pixel of said sample image in response to said sample speed.

29-31. (canceled)

32. The apparatus of claim 1, wherein each beam of said array of beams has a diffraction-limited beam width.

33. The apparatus of claim 1, wherein a spacing between beams of said array of beams is proportional to said inter-element gap.

34. A time-resolved luminescence imaging method, comprising:
generating an array of beams;
scanning said array of beams with respect to a sample, along a single scanning axis; and
detecting emissions generated by said sample in response to said array of beams with an array of detector elements of a detector assembly, adjacent detector elements being spaced apart by an inter-element gap.

35-66. (canceled)

67. A computer configured to control an imaging apparatus to perform the method of any one of claim 34.

68-124. (canceled)
